(19) United States
(12) Patent Application Publication
(10) Pub. No.: US 2011/0184509 A1
(43) Pub. Date: Jul. 28, 2011

(54) DUAL SHEATH ASSEMBLY AND METHOD OF USE

(75) Inventors: Randolf Von Oepen, Los Altos, CA (US); Travis R. Yribarren, Campbell, CA (US)

(73) Assignee: ABBOTT LABORATORIES, Santa Clara, CA (US)

(21) Appl. No.: 12/694,821

(22) Filed: Jan. 27, 2010

(51) Int. Cl. A61F 2/84

(52) U.S. Cl. 623/1.23

(57) ABSTRACT

A sheath assembly is provided for protecting a stent mounted on a catheter. An inner tubular member is positioned over the stent without longitudinal movement of the inner tubular member along the stent surface thereby eliminating the possibility of scraping or scratching a drug coating or polymer coating on the stent surface or balloon surface. An outer tubular member slides over the inner tubular member to firmly compress it onto the stent for further protection. In use, the outer tubular member is removed from over the inner tubular member so that the two half-cylindrical portions of the inner tubular member can fall away from the stent without longitudinal movement along the stent surface.

### BACKGROUND

The invention relates to stent delivery systems, which are used to implant a stent into a patient's body lumen to maintain the patency thereof. More particularly, the present invention relates to a sheath assembly that is mounted over a stent on a catheter for deploying the stent in a body lumen.

Stents are generally cylindrically-shaped devices which function to hold open and sometimes expand a segment of a blood vessel or other body lumen such as a coronary artery. They are also suitable to support and hold back a dissected arterial lining that can occlude the fluid passageway. Stents also are useful in maintaining the patency of a body lumen, such as a coronary artery, after a percutaneous transluminal coronary angioplasty (PTCA) procedure. The delivery and deployment of stents in coronary arteries are well known in the art and various types of catheters are used, along with guidewires, to position and implant the stent in the artery.

Presently, stents sometimes are coated with polymer coatings that may include a drug for the purpose of reducing the likelihood of the development of restenosis. The polymer coating and the drug can be easily scratched or removed if the drug coating comes into contact with any outside agency. It is important to maintain the integrity of the stent surface in order to ensure the clinical efficacy of the drug coating. Traditionally, in order to protect the stent surface, a sheath is applied over the stent, which is then packaged until it is removed from the package by the physician in preparation for use. When the sheath is applied to or removed from the stent, it slides over the stent and may cause damage to the polymer coating or the drug coating.

It is therefore important to improve the existing sheath assemblies to protect the polymer and drug coatings on the stent during both the application and removal of the sheath assembly from the stent surface. The present invention satisfies these needs.

### SUMMARY OF THE INVENTION

The present invention is directed to a sheath assembly for protecting a stent mounted on a catheter. In one embodiment, the sheath assembly has an outer tubular member and an inner tubular member where the inner tubular member has at least two longitudinal slits that extend from a proximal end of the inner tubular member to a point near the distal end of the inner tubular member. In a pre-assembly state, the inner tubular member may be hinged where the longitudinal slit terminates near the distal end of the inner tubular member. Thus, the inner tubular member has a clamshell-type configuration so that it can essentially open and close radially outwardly onto the stent mounted on a catheter, without the possibility of sliding longitudinally along the stent thereby preventing any scraping or scratching movement on the stent surface, and specifically on the drug coated polymer coating on the stent surface. The inner tubular member is positioned over the stent by moving the generally half-cylindrical portions formed by the longitudinal slits radially inwardly to in essence clamp down on the stent. Thereafter, the outer tubular member slides over the inner tubular member in a tight-fitting manner so as to slightly compress the inner tubular member onto the stent. The distal end of the inner tubular member extends out of the outer tubular member and is cut off by any appropriate means (e.g., with a laser, knife, scalpel, razor blade, scissor, etc.). Thus, the inner tubular member then becomes two half-cylindrical portions that are unattached to each other and which are compressed onto the stent by the outer tubular member. The entire catheter assembly, including the stent and the protective sheath assembly are then packaged in a known manner. In use, the physician removes the catheter assembly from the packaging, and pulls the outer tubular member in a distal direction to slide it distally off of the inner tubular member. Thereafter, the inner tubular member is removed from the stent as the half-cylindrical portions of the inner tubular member fall away from the stent without any longitudinal movement along the stent surface.

Other features and advantages of the present invention will become more apparent from the following detailed description of the invention when taken in conjunction with the accompanying drawings.

### BRIEF DESCRIPTION OF THE DRAWINGS

FIG. 1 is a side view, partially in section, depicting a delivery catheter and stent, with a prior art sheath covering the stent.

FIG. 2 is a side view of the sheath assembly including the inner tubular member having longitudinal slits and being covered by the outer tubular member.

FIG. 3 is a side view depicting the stent delivery catheter with the inner tubular member being mounted on the stent.

FIG. 4 is a side view, partially in section, depicting the catheter assembly including the outer tubular member sliding over the inner tubular member.

FIG. 5 is a side view, depicting the inner and outer tubular members mounted on the catheter assembly and the distal portion of the inner tubular member being cut off.

FIG. 6 is a side view, partially in section, showing the catheter assembly with the stent mounted thereon and the inner tubular member and outer tubular member being completely removed from the catheter assembly.

FIG. 7 is a side view depicting the inner tubular member having a single longitudinal slit extending from the distal to the proximal end.

FIG. 8 is a cross-sectional view taken along the lines 8-8 of FIG. 7, depicting a transverse cross-sectional view of the inner tubular member.

FIG. 9 is a side view, partially in section, depicting a catheter assembly with a stent mounted thereon and the inner tubular member being applied over the stent.

FIG. 10 is a side view depicting the catheter assembly with the inner tubular member covering the stent (not shown) and the outer tubular member sliding over the inner tubular member.

### DETAILED DESCRIPTION OF THE PREFERRED EMBODIMENTS

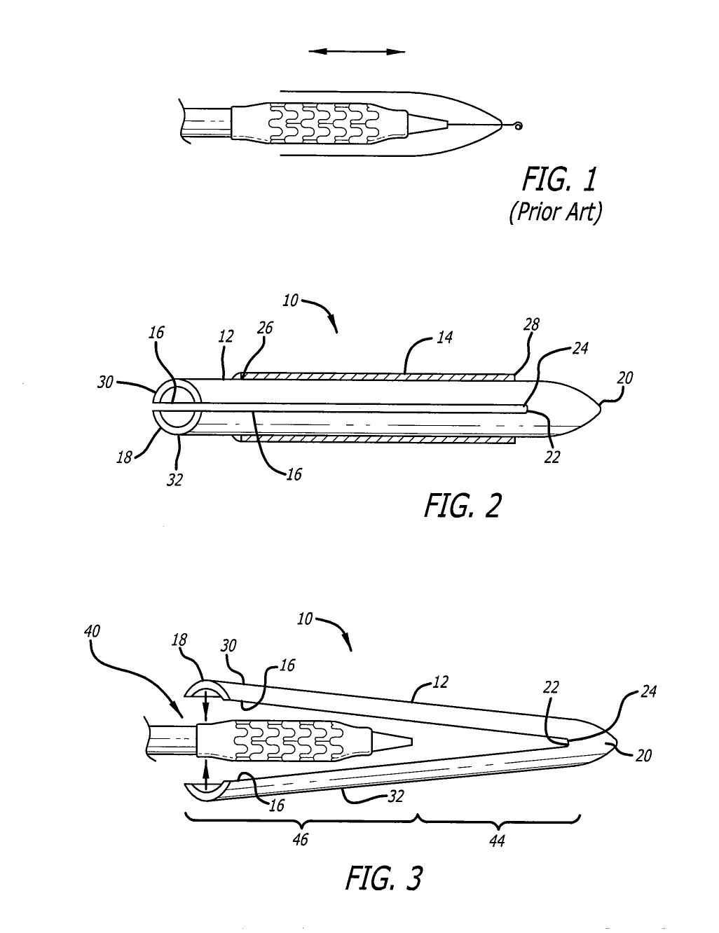

Prior art stent delivery systems, such as the one shown in FIG. 1, typically have an intravascular stent mounted on a balloon catheter with a removable sheath covering the stent for protection. The protective sheath slides longitudinally over the stent for protecting the stent during packaging and delivery to the end user. When the physician is ready to deploy the stent, the sheath is removed by pulling it longitudinally off of the stent in a distal direction. Since the protective sheath fits tightly on the stent in order to protect it, it can actually scratch or scrape along the stent surface and cause damage to the stent. This is particularly damaging when the stent is coated with a therapeutic agent or drug which may also include a polymer which is infused with the therapeutic agent. If the drug or therapeutic agent is scratched or scraped when the protective sheath is placed over the stent or removed from the stent, it can adversely affect the efficacy of the drug eluting from the stent when it is deployed in a coronary artery. The present invention solves the problem of sliding the protective sheath on and off the catheter assembly so that the stent with its drug coating is not scratched or scraped during the process.

Stent retention, surface integrity, and crossing profile are characteristics of a stent delivery system that are important to positive clinical outcomes. Each of these characteristics may be impacted by the packaging sheath of a stent delivery system. For example, a packaging sheath can constrain the dimensions of the stent and balloon during the storage process, which impacts the overall profile and stent retention since it insures that the stent does not expand during storage. In theory, it is possible for a stent and balloon to expand slightly during storage due to residual stresses or fluctuations in humidity and temperature. Even slight expansions of the stent may negatively affect stent retention. Further, the packaging sheath protects the stent surface during storage, but can also be a factor in scratching the stent or balloon surface during removal. Existing sheaths are oversized cylindrical shapes such as the one depicted in FIG. 1. In order to slide the prior art sheath onto the stent and catheter, there necessarily must be a gap between the sheath and the stent so that the sheath does not constrain the stent to its original profile. Also, removal of the sheath applies a tangential load that can both scratch the stent surface and pull the stent axially. Thus, there is a need for a packaging sheath that constrains the stent to substantially the same profile and yet does not apply a tangential load (longitudinally) to the stent during the mounting and removal of the sheath.

In keeping with the present invention, as shown in FIGS. 2-6, a sheath assembly 10 is provided for protecting a stent mounted on a catheter and to avoid the longitudinal movement of a sheath sliding over the stent surface. More particularly, sheath assembly 10 includes an inner tubular member 12 and an outer tubular member 14. The inner tubular member 12 fits over a stent and the outer tubular member 14 slidingly engages and extends over the inner tubular member 12. As shown in FIG. 2, the inner tubular member 12 has a pair of longitudinal slits 16 extending from the proximal end 18 of the inner tubular member toward the distal end 20 of the inner tubular member. By “longitudinal” it is meant that the slits extend lengthwise along the tubular member, but not necessarily parallel to the longitudinal axis of the tube. The longitudinal slits 16 terminate at a point 22 that is near the distal end 20 of the inner tubular member 12, but not at the distal end. Further, a hinge portion 24 is formed at the terminal distal ends of the longitudinal slits at point 22. The outer tubular member 14, which is configured to slide over the inner tubular member, has a proximal end 26 and a distal end 28. The longitudinal slits 16 in the inner tubular member result in a first half-cylindrical portion 30 and a second half-cylindrical portion 32. In conjunction with the hinge portion 24, the first half-cylindrical portion 30 and the second half-cylindrical portion 32 open and close similar to a clamshell opening and closing. Importantly, the first half-cylindrical portion 30 and the second half-cylindrical portion 32 move radially outwardly or inwardly so that there is no longitudinal movement of the inner tubular member on the surface of the stent, thereby removing any likelihood that the inner tubular member will scratch or scrape the drug or polymer surface on the stent.

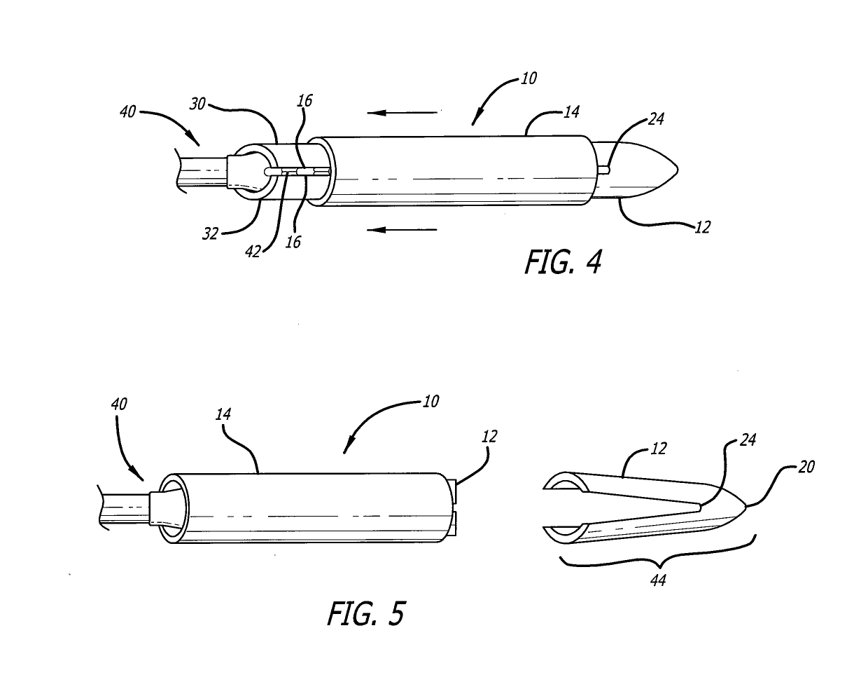

With reference to FIGS. 3 and 4, the sheath assembly 10 is mounted on the distal portion of a balloon catheter 40. Only the distal portion of the balloon catheter 40 is shown, as balloon catheters are well known in the art and can have numerous configurations. A stent 42 is mounted on the balloon portion of the balloon catheter, but the stent 42 is not visible since it is covered by the inner tubular member 12. Still referring to FIGS. 3 and 4, the inner tubular member 12 has been placed over the stent 42 by moving the first half-cylindrical portion 30 and the second half-cylindrical portion 32 radially inwardly until they firmly are positioned over the stent 42. As set forth above, the hinge portion 24 permits the first and second half-cylindrical portion 30,32 to move in a clamshell-like movement in order to open and close onto the stent. Further, the outer tubular member 14 is pushed over the inner tubular member 12 after it is positioned over the stent. A proximal flared portion (not shown) on the first and second half-cylindrical portions may be used to aid assembly. The outer tubular member fits snugly onto the inner tubular member so that it compresses the inner tubular member onto the stent 42. Although the outer tubular member 14 slides over the inner tubular member 12, this does not cause any scraping or scratching on the drug coating on the stent since the inner tubular member 12 covers and protects the stent from the longitudinal movement as the outer tubular member slides over the inner tubular member.

Referring to FIG. 5, the sheath assembly 10 is assembled on the balloon catheter 40 and over the stent 42 in order to protect the stent and any coating on the balloon and stent during packaging. As can be seen, the inner tubular member 12 has a distal portion 44 that includes a length of the inner tubular member having hinge portion 24. The distal portion 44 of the inner tubular member 12 is removed by cutting away the distal portion by any appropriate means including using a laser, knife, scalpel, razor blade, or scissors. The remaining proximal portion 46 of the inner tubular member covers and protects the stent. Thereafter, the inner tubular member 12 is comprised of the first half-cylindrical portion 30 and the second half-cylindrical portion 32, which are now unconnected to each other. The first and second half-cylindrical portions 30,32 still protect the stent and the coating on the stent and/or balloon since they cover the stent and balloon, with the outer tubular member 14 remaining snugly over the first and second half-cylindrical portions.

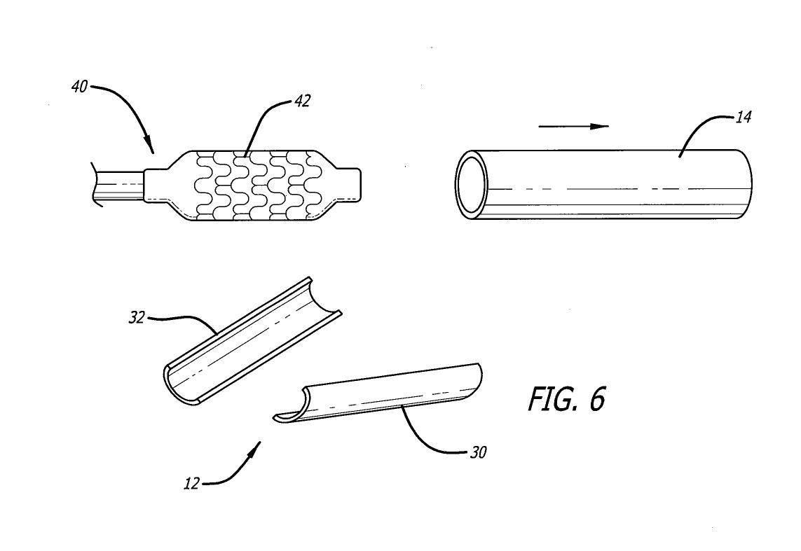

As can be seen in FIG. 6, the sheath assembly 10 is being removed from the balloon catheter 40 and stent 42. The outer tubular member 14 is withdrawn distally from over the inner tubular member 12. After the outer tubular member 14 is completely withdrawn from the inner tubular member 12, the first half-cylindrical portion 30 and the second half-cylindrical portion 32 will simply fall away from the stent 42 and balloon catheter 40. Importantly, as the first half-cylindrical portion 30 and the second half-cylindrical portion 32 fall away from the stent, they do not scrape or scratch the stent surface or any drug coating or polymer coating on the stent surface or any coating on the balloon.

Alternatively, and with reference to FIGS. 2-6, the longitudinal slits 16 extend from the proximal end 18 to the distal end 20 of the inner tubular member 12. This results in first and second half-cylindrical portions 30,32 that are unconnected to each other. In this embodiment, the first and second half-cylindrical portions 30,32 are pressed over the stent 42 and then the outer tubular member 14 is pushed over the first and second half-cylindrical portions with a slight compressive force so that they press onto the stent. When the balloon catheter 40 is being prepared for use, the outer tubular member 14 is removed by sliding it off of the first and second half-cylindrical portions 30,32, which are then free to simply fall away from the stent 42 without any longitudinal movement over the stent surface or the drug or polymer coatings on the stent surface. In this embodiment, the first and second half-cylindrical portions can be formed by making the longitudinal slits 16 in the inner tubular member 12 or by pre-forming the half-cylindrical portions in an injection mold or by similar molding processes.

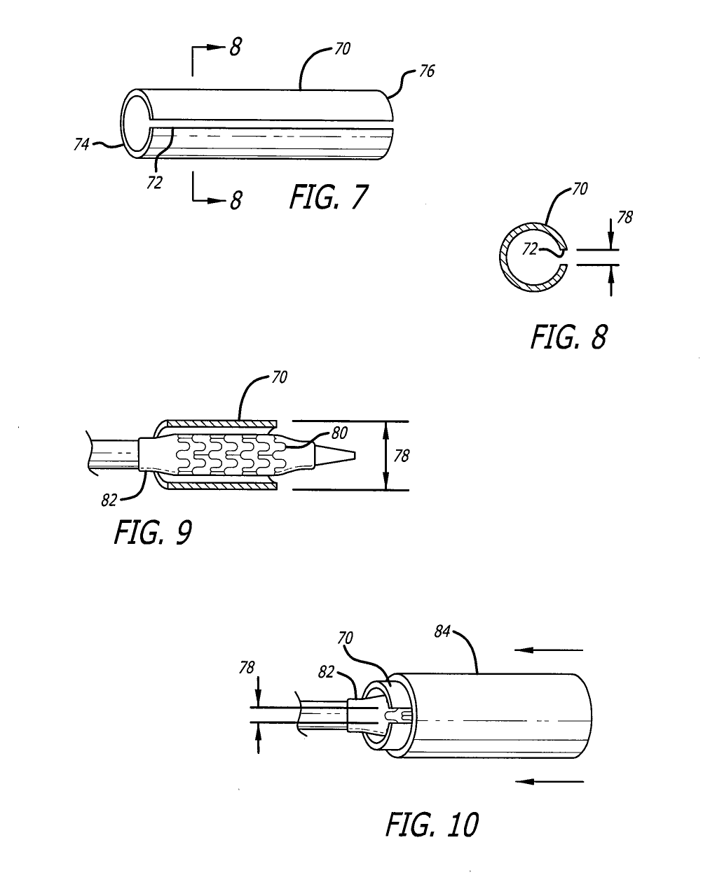

In another embodiment, as shown in FIGS. 7-10, the inner tubular member 70 includes a longitudinal slit 72 that extends from the inner tubular member proximal end 74 to the distal end 76. The longitudinal slit 72 can be a single slit without removing material, or can have some material removed so that there is a gap 78 such as that shown in FIG. 8. Preferably, inner tubular member 70 is formed from a highly elastic polymer material so that the gap 78 can be spread open to extend over a stent on a balloon catheter assembly, such as that shown in FIG. 9. Thus, the inner tubular member 70 is spread apart along the longitudinal slit 72 to form a large gap 78 (see FIG. 9) in order to extend over stent 80 mounted on the balloon catheter 82. As the inner tubular member 70 is closed over the stent 80, there is no sliding or scraping motion of the inner tubular member over the stent so that any polymer or drug coating on the stent is not scratched or scraped as the inner tubular member is mounted on the stent. As shown in FIG. 10, the inner tubular member 70 completely covers the stent so that the gap 78 is very small. An outer inner tubular member 84 slidingly engages the inner tubular member 70 and provides a slight compressive force onto the inner tubular member in order to reduce the size of gap 78 as well as slightly compress the inner tubular member onto the stent in order to protect the stent during packaging. Since the outer tubular member 84 slides over the inner tubular member, it does not come in contact with the stent thereby eliminating any possibility of scratching or scraping the polymer or drug surface on the stent. Prior to deploying the stent, the outer tubular member 84 slides off of the inner tubular member 70 and the gap 78 is expanded thereby opening the inner tubular member so that it can be removed from the stent without any scraping or sliding movement on the stent surface.

The sheath assembly embodiments shown in FIGS. 2-6 can be formed from a number of polymer materials that are well known in the art such as LLDFE, HDPE and LDPE. For example, the inner tubular members can be formed from an elastomeric polymer such as urethane, rubber, latex and TECOFLEX®. The outer tubular member preferably is formed of a more rigid material than the inner tubular member and can be formed of such materials as polyether ether ketone (PEEK), a rigid plastic such as acrylonitrile-butadiane-styrene (ABS) or polyvinyl chloride (PVC) or from a fiber or braid reinforced tube from any combination of these materials, all of which will enhance the pushability of the outer tubular member over the inner tubular member. Depending upon the particular application, either of the inner and outer tubular members may be flexible or more rigid and be formed from polyethylene materials such as LLDFE, HDPE or LDPE as examples.

Although preferred alternative embodiments have been described and illustrated, the invention is susceptible to modifications and adaptations within the ability of those skilled in the art and without the exercise of inventive faculty. Thus, it should be understood that various changes in form, detail, and usage of the present invention may be made without departing from the spirit and scope of the invention. Accordingly, it is not intended that the invention be limited, except as by the appended claims.

What is claimed:

1. A sheath assembly for protecting a stent mounted on a catheter, comprising:
a sheath assembly having an outer tubular member and an inner tubular member;
the inner tubular member having two longitudinal slits thereby forming a first half-cylindrical portion that is unattached to a second half-cylindrical portion; and
the outer tubular member extending over at least a portion of the inner tubular member.

2. The sheath assembly of claim 1, wherein the outer tubular member is configured for slidable movement over the inner tubular member.

3. The sheath assembly of claim 2, wherein the outer tubular member has an inner diameter and an outer diameter and the inner tubular member has an inner diameter and an outer diameter, the inner diameter of the outer tubular member being greater than the outer diameter of the inner tubular member.

4. The sheath assembly of claim 2, wherein the outer tubular member is sized to compress the inner tubular member when the outer tubular member slides over the inner tubular member.

5. The sheath assembly of claim 1, wherein the inner tubular member has a proximal portion having a length sufficient to cover a stent, and a removable distal portion that extends distally beyond the stent.

6. The sheath assembly of claim 5, wherein the removable distal portion of the inner tubular member includes a hinge portion.

7. The sheath assembly of claim 1, wherein the inner tubular member and the outer tubular member are formed from a polymer material, the polymer material of the outer tubular member having a different rigidity than the polymer of the inner tubular member.

8. The sheath assembly of claim 1, wherein the inner tubular member is formed from an elastomer or polymer, including any of urethanes, rubber, latex, LLDFE, HPPE or LDPE.

9. A sheath assembly for protecting a stent mounted on a catheter, comprising:
a sheath assembly having an outer tubular member and an inner tubular member;
the inner tubular member having two longitudinal slits generally diametrically opposed to form a first half-cylindrical portion that is unattached to a second half-cylindrical portion; and
the outer tubular member configured for slidable movement over the inner tubular member so that the outer tubular member applies a slight compressive force on the inner tubular member.

10. The sheath assembly of claim 9, wherein the outer tubular member is configured for slidable movement over the inner tubular member.

11. The sheath assembly of claim 10, wherein the outer tubular member has an inner diameter and an outer diameter and the inner tubular member has an inner diameter and an outer diameter, the inner diameter of the outer tubular member being greater than the outer diameter of the inner tubular member.

12. The sheath assembly of claim 10, wherein the outer tubular member is sized to compress the inner tubular member when the outer tubular member slides over the inner tubular member.

13. The sheath assembly of claim 9, wherein the inner tubular member has a proximal portion having a length sufficient to cover a stent, and a removable distal portion that extends distally beyond the stent.

14. The sheath assembly of claim 13, wherein the removable distal portion of the inner tubular member includes a hinge portion.

15. The sheath assembly of claim 9, wherein the inner tubular member and the outer tubular member are formed from a polymer material, the polymer material of the outer tubular member having a different rigidity than the polymer of the inner tubular member.

16. The sheath assembly of claim 9, wherein the inner tubular member is formed from an elastomer or polymer, including any of urethanes, rubber, latex, LLDFE, HDPE or LDPE.

17. A sheath assembly for protecting a stent mounted on a catheter, comprising:
a sheath assembly having an outer tubular member and an inner tubular member;
the inner tubular member having a single longitudinal slit; and
the outer tubular member extending over at least a portion of the inner tubular member.

18. The sheath assembly of claim 17, wherein the outer tubular member is configured for slidable movement over the inner tubular member.

19. The sheath assembly of claim 18, wherein the outer tubular member has an inner diameter and an outer diameter and the inner tubular member has an inner diameter and an outer diameter, the inner diameter of the outer tubular member being greater than the outer diameter of the inner tubular member.

20. The sheath assembly of claim 18, wherein the outer tubular member is sized to compress the inner tubular member when the outer tubular member slides over the inner tubular member.

21. The sheath assembly of claim 17, wherein the inner tubular member has a generally C-shaped transverse cross-section so that the inner tubular member can open and close over a drug coated stent without scraping the drug coated stent surface.

22. A method for mounting a sheath assembly onto a stent delivery catheter, comprising:
providing a balloon catheter assembly with a drug coated stent mounted on a balloon portion of the catheter assembly;
providing a sheath assembly having an inner tubular member and an outer tubular member;
forming diametrically opposed longitudinal slits in a proximal portion of the inner tubular member;
mounting the inner tubular member onto the stent without any longitudinal movement between the inner tubular member and the stent; and
sliding the outer tubular member over the inner tubular member so that the outer tubular member slightly compresses the inner tubular member onto the stent.

23. The method of claim 22, wherein the longitudinal slits terminate to form a hinge portion in a removable distal portion of the inner tubular member.

24. The method of claim 23, wherein the hinge portion allows the proximal portion of the inner tubular member to open and close over the stent in a clamshell-like movement with no longitudinal movement relative to the stent.

25. The method of claim 23, wherein the removable distal portion of the inner tubular member, including the hinge portion, is cut away from the proximal portion of the inner tubular member.

26. The method of claim 25, wherein the distal portion of the inner tubular member is cut away from the proximal portion by any of a laser, knife, razorblade, scissor, or other cutting instrument.
